(19) United States
(12) Patent Application Publication
(10) Pub. No.: US 2026/0240802 A1
(43) Pub. Date: Aug. 20, 2026

(54) USE OF MYRIOCIN AS ADJUVANT IN GLAUCOMA SURGERY

(71) Applicants: Luca Mario Rossetti, Milan (IT); Anna Caretti, Milan (IT); Leonardo Colombo, Milan (IT); Dario Romano, Milan (IT)

(72) Inventors: Luca Mario Rossetti, Milan (IT); Anna Caretti, Milan (IT); Leonardo Colombo, Milan (IT); Dario Romano, Milan (IT); Michele Vittorio DEI CAS, Milan (IT)

(73) Assignees: Luca Mario Rossetti, Milan (IT); Anna Caretti, Milan (IT); Leonardo Colombo, Milan (IT); Dario Romano, Milan (IT)

(21) Appl. No.: 19/161,639

(22) PCT Filed: Mar. 4, 2024
(86) PCT No.: PCT/IB2024/052052

§ 371 Date: Sep. 2, 2025

(30) Foreign Application Priority Data
Mar. 3, 2023 (IT) 102023000003891

(51) Int. Cl. A61K 31/201; A61P 27/02

(52) U.S. Cl. CPC A61K 31/201; A61P 27/02

(57) ABSTRACT

The present invention relates to the compound myriocin and compositions comprising it for use in the surgical treatment or to assist in the surgical treatment of glaucoma, in particular for use in the treatment or to assist in the treatment of a glaucomatous subject undergoing surgical therapy of glaucoma.

### FIELD OF THE INVENTION

The present invention relates to myriocin and to compositions containing it for use in the treatment or to assist in the treatment of glaucoma.

### STATE OF ART

Glaucoma represents the first cause of irreversible blindness in the world. The known main risk factor is represented by the high intraocular pressure which causes a progressive degeneration of the retinal ganglion cells and of the nerve fibres originating therefrom, with consequent deterioration of the field of view until blindness.

The main objective of the conventional anti-glaucomatous therapies is the lowering in the intraocular pressure. The first line therapy generally consists in using hypotonizing topical drugs or mininvasive laser treatments. In case of failure of the first line therapy or, in case of particular forms of glaucoma, the surgical therapy represents the main indication. In particular, the most commonly performed interventions are trabeculectomy and the implantation of draining devices, which allow to create an accessory route to the outflow of aqueous humour.

In 2020, approximately 80 million people in the world resulted to be suffering from glaucoma and it is estimated that in 2040 the number of suffering people will increase to 111 million. The average age at diagnosis is 55 years and 5.3% of suffering patients undergo surgery within 5 years from diagnosis. In case of failure of a surgical intervention, it is possible to repeat the same intervention in a different site or to have recourse to a different surgical technique; however, the re-intervention is linked to a higher failure rate.

In case of surgical therapy of glaucoma, the main cause of failure is represented by fibrosis and healing of the surgical site, which determines a progressive obstruction of the accessory route create during the intervention, with consequent ocular hypertonicity.

Currently, the drug which has demonstrated to be the most effective in contrasting postoperative fibrosis is mitomycin C, although it was not approved for such use. After introducing such drug within the intraoperative therapy, the percentage of failure of the surgical therapy dropped from 24-74% after 4 years to 23-51% after 5 years.

Mitomycin C, originally used in the oncology field as antineoplastic, is exploited in the surgery of glaucoma thanks to its capability of inhibiting the replication of fibroblasts, and then fibrosis, in the application site.

However, different histological studies have highlighted that the ocular area treated with mitomycin C often shows a reduction, until the complete absence, of vascular structures, with consequent risk of hypoxic-ischemic damage, whereas the surrounding area shows an increased density of fibroblasts and a vascularization increase. The application of mitomycin C can further reduce the defensive response of the immune system in loco, with consequent increased sensibility to pathogen agents and risk of endophthalmitis even eleven years after application.

The adverse effects encountered with the application of mitomycin C have demonstrated to be dependent on the dose and time of exposure to the drug, with consequent reduction in clinical handling of such substance and impossibility to perform repeated treatments. Due to these features, the use of mitomycin C is almost always reserved for the intraoperatory phase of glaucoma treatment and currently is limited to an off-label use. Although its use is extremely widespread in the world, currently there is a considerably variability in the concentrations and exposure time and a greater standardization is necessary to reduce complications and to maximize its efficacy.

In such scenario, it results then still evident the need for developing therapeutic approaches having greater clinical efficacy which are capable of improving significantly the treatment of glaucoma.

### SUMMARY OF THE INVENTION

Studies performed by the present inventors on an in vitro experimental model of human dermal fibroblasts activated with TGFβ (Transforming Growth Factor β), have surprisingly demonstrated that the administration of myriocin allows to explicate anti-fibrotic efficacy comparable to that of the drug mitomycin C, however guaranteeing a better tolerability at cellular level.

Myriocin is a natural compound derived from fungus Mycelia sterilia. It acts as powerful inhibitor of serine-palmitoyl transferase, enzyme which adjusts the key stage, limiting the synthesis of sphingolipids, by locking the production of ceramide. Myriocin was tested in cellular and animal models of cystic fibrosis, ischemia-reperfusion and retinitis pigmentosa for its anti-inflammatory and anti-apoptotic properties, demonstrating a good efficacy without highlighting significant side effects. The anti-fibrotic activity of myriocin was described in literature in an animal model of pulmonary fibrosis induced by radiations and in an animal model of non-alcoholic steatosis, but there is no evidence of using the substance as adjuvant in the surgical treatment of glaucoma.

As clearly illustrated in the experimental section reported in the present application, the authors of the present invention found that the administration of myriocin allows to contrast the pro-fibrotic effects mediated by TGFβ by locking the synthesis of ceramide; in particular they demonstrated that myriocin:
inhibits the enzyme serine-palmitoyl transferase by reducing inflammation and fibrogenesis induced by TGFβ;
restores the proteolytic damage of the extracellular matrix by reducing the expression of MMP-9 (Matrix metalloproteinase 9), enzyme that degrades the proteins of the extracellular matrix; the control of activity of metalloproteases by specific inhibitors contributes in modulating the fibrotic process;
reduces the expression of αSMA (α Smooth Muscle Actin) and the accumulation of proteins of the extracellular matrix (fibronectin and collagen), both of them induced by TGFβ.

Thanks to the significant anti-fibrotic activity found in the performed experimental tests, the present invention then provides, for the first time, the use of myriocin as effective solution in the treatment or to assist in the surgical treatment of glaucoma.

Therefore, the invention relates to myriocin for use in the treatment of glaucoma or to assist in the surgical treatment of glaucoma.

The invention also relates to compositions for use in the treatment of glaucoma or to assist in the surgical treatment of glaucoma comprising myriocin and one or more carriers and/or diluents and/or excipients.

The present invention involves a series of advantages in the therapy against glaucoma, one of the main causes of blindness in the world:
the possibility of explicating action in assisting in the surgery of glaucoma, for example in pre-, intra- and/or post-surgery phase, by guaranteeing an anti-inflammatory and anti-cicatricial effect.
the possibility of obtaining a good safety and tolerability profile, even after repeated administrations, for example for at least 3 days, feature which makes the use of myriocin or compositions comprising it extremely easy to handle.

Other advantages, features and use modes of the present invention will result evident from the following detailed description of some embodiments, shown by way of example and not for limitative purposes.

### BRIEF DESCRIPTION OF FIGURES

FIG. 1. Migratory and reparative capability of the damage by fibroblasts undergoing anti-fibrotic treatment with Myriocin or Mitomycin C.

The images, obtained at the optical microscope (40× enlargement), represent the centrally damaged cellular carpet in the experimental groups: control without treatment (ctr) at T0, after 24 (T1) and 48 hours (T2); treated with Myriocin (Myr) 24 (T1) and 48 hours (T2) after start of treatment (T0); treated with Mitomycin C (Mito) 24 (T1) and 48 hours (T2) after start of treatment (T0). The black lines designate the width of the damage inside thereof the fibroblasts treated with Myriocin and Mitomycin migrate only partially with respect to the not treated control. The fibroblasts treated with Mitomycin C show a progressive morphological change (from tapering to rounded) between T1 and T2 with respect to the fibroblasts treated with Myriocin.

FIG. 2. Expression of the transcripts of genes involved in the fibrotic process.

Expression of the transcript (mRNA) of αSMA, CTGF, MMP-9 evaluated by RT-PCR, in fibroblasts: control (ctr); stimulated with TGFβ (TGFβ); pretreated 1 hour with Myriocin and for the subsequent 24 hours in combination with TGFβ (Myr/TGFβ); pretreated 5 minutes with Mitomycin C, washed and treated for the subsequent 24 hours with TGFβ (Mito/TGFβ); pretreated 1 hour with Myriocin and for the subsequent 24 hours with Myriocin (Myr); pretreated 5 minutes with Mitomycin C, washed and for the subsequent 24 hours with culture medium (Mito). The results represent the average±standard error of 5 experiments and they are represented as variation with respect to the control (ratio between the expression in the various groups and the control). The control is set equal to 1 and designated in figure with the dotted line. The statistical analysis was performed by one-way ANOVA test, followed by a post-test (Sidak test) when significant (p<0.05). p=0.0003, p=0.0005 and p=0.023 they refer to the significance of the TGFβ group with respect to the group ctr. ****, p<0.0001; **, p<0.005; *, p<0.01.

FIG. 3. Protein expression of αSMA in response to the treatment with Myriocin or Mitomycin.

The image is the most representative of three experiments; the protein band has a weight of approximately 42 KDa.

FIG. 4: Ctrl: not treated fibroblasts; TGFβ: fibroblasts treated 24 hours with TGFβ 5 ng/ml; TGFβ/Myr: fibroblasts pre-treated 1 hour with Myriocin 50 UM and for subsequent 24 hours in combination with TGFβ 5 ng/ml; TGFβ/Mito: fibroblasts pre-treated 5 minutes with Mitomycin 50 UM, and for subsequent 24 hours with TGFβ 5 ng/ml in absence of Mitomycin.

### GLOSSARY

The terms used in the present description, unless otherwise indicated, have the meaning commonly used in the state of art.

The expression “to assist in the treatment of glaucoma”, in the context of the present invention relates to the capability of myriocin or a composition comprising it according to any one of the variants described in the present description, to join to a drug or a base therapy, in particular a therapy of surgical treatment of glaucoma, to reinforce or complete the action thereof.

The expressions “surgical therapy”, “surgical treatment therapy”, “surgical method”, “surgical treatment method” or “surgical operation” can be used interchangeably in the context of the present invention.

The expressions “glaucomatous subject” and “subject suffering from glaucoma” can be used interchangeably in the context of the present invention.

According to a preferred aspect of the invention, under “therapeutically effective amount” an amount of myriocin or of a composition comprising it according to any one of the variants described in the present description is meant, which allows to obtain a decrease in or inhibition of the replication of the fibroblasts, and then fibrosis, in the application site of the compound or of the composition at ocular level.

Under the expression “carriers, diluents and/or excipients suitable for the preparation of ophthalmic compositions”, in the context of the present invention, carriers, diluents and/or excipients tolerated by the eye are meant.

In any point of the present description and of claims, the term “comprising” can be replaced by “consisting of”.

### DETAILED DESCRIPTION

A first aspect of the present invention relates to myriocin for use in the treatment of glaucoma or to assist in the treatment of glaucoma. In particular, an aspect of the present invention relates to myriocin for use in the surgical treatment of glaucoma or in assisting in the surgical treatment of glaucoma.

According to the present invention, myriocin, acquired or formulated according to any one of the protocols known to a person skilled in the art, can be used in the treatment or to assist in the treatment, and in particular before, during and/or after the surgical treatment, of all different forms of glaucoma, such as, for example, congenital glaucoma, open-angle glaucoma, closed-angle glaucoma, or glaucoma without hypertension in a subject, preferably a human subject. According to an aspect of the invention, myriocin is administered to a subject suffering from glaucoma, in particular a human subject, in a therapeutically effective amount.

The present invention also relates to a composition for use in the treatment of glaucoma or to assist in the treatment of glaucoma, wherein said treatment in particular is surgical treatment of glaucoma, comprising myriocin and one or more carriers, and/or diluents and/or excipients.

According to an aspect of the invention, a composition according to any one of the variants illustrated in the present description or claims is a pharmaceutical composition.

According to an aspect of the invention, a composition according to any one of the variants illustrated in the present description or claims is an ophthalmic composition.

Preferably, a composition according to any one of the embodiments of the present invention comprises myriocin and one or more carriers, diluents and/or excipients suitable for the preparation of ophthalmic compositions or one or more pharmaceutically acceptable carriers, diluents and/or excipients.

Carrier compounds, diluents and/or excipients which can be used in a composition according to any one of the embodiments described in the present description can be selected among stabilizers, preservatives, solvents, pH regulators, isotonia regulators, chelating agents, cryoprotective agents, diluting agents, binding agents, antioxidants, surfactants or combination thereof.

Examples of excipients or carriers which can be used in a composition according to the invention are polysorbate 80, polyethylene glycol, dextran, as well as any compound suitable to increase bioavailability, stability and/or tolerability of myriocin.

According to an additional aspect of the present invention, a composition according to any one of the embodiments described in the present description can include protective compounds which can facilitate the transportation and/or specific release of myriocin, slightly soluble lipophilic substance, at ocular level. Such compounds can include any system of pharmacological transportation known in the field, for example biocompatible polymers, nano- or micro-particle systems, liposomes, nanostructured materials, photosensitive capsules, nanoparticles, dendrimers, hybridized vesicles, cationic lipids, solid lipid nanoparticles (SLN), nanostructured lipid carriers, solid lipid microparticles or combinations thereof.

According to a preferred aspect, a composition according to any one of the embodiments described in the present description comprises solid lipid nanoparticles as carriers.

The solid lipid nanoparticles are colloidal systems, stabilized by surfactants, with sizes preferably comprised between 10 and 1000 nm and they can be prepared by using different types of lipids, thereamong pure triglycerides, mixed complexes of glycerides or waxes. Among the most used surfactants there are phospholipids, poloxamers and polysorbates. As a person skilled in the art knows, the sizes and surface charge of the solid nanoparticles can be modulated depending upon the needs, by suitable selection of the lipids and surfactants. Lipids suitable to be used for the preparation of lipidic nanoparticles such as ophthalmic carriers are lipids which allow an optimum solubilization of myriocin, such as, for example triglycerides, capric/caprylic triglycerides, propylene glycol dicaprylocaprate, dyglicerides, monoglycerides, aliphatic alcohols, the fatty acids with chains C₁₀-C₁₂, cholesterol and esters thereof.

According to a particularly preferred aspect, a composition according to any one of the embodiments described in the present description comprises solid lipid nanoparticles with a diameter comprised between 40 and 200 nm as carriers.

The use of solid lipid nanoparticles has several advantages: it allows a modified release of myriocin; it improves bioavailability of myriocin slightly soluble in water; it allows applications for different administration routes; it confers protection of the compound from degradation processes of chemical, physical and enzymatic type; it offers high drug entrapment efficiency; it guarantees low production costs; it has high biodegradability and good tolerability. Due to the biocompatibility and mucoadhesive properties, SLNs show a greater interaction with the mucous membrane of the eye and then prolong the residence time of myriocin at ophthalmic level and allow it to overcome the corneal barrier. The medicinal substances under form of SLN can penetrate effectively in the epithelium, thanks to their lipophilic properties.

To prevent contaminations, any composition according to the present invention preferably comprises one or more preservatives with antimicrobic activity, such as benzalkonium chloride (abbreviated with acronym BAK).

The concentration of myriocin in a composition according to any one of the embodiments and application described in the present description is preferably comprised in the range between 0.1 and 5 mg per gram or mL of composition, preferably between 0.1 and 1 mg per gram or mL of composition, still more preferably it is equal to 0.68 mg per mL or equal to 1 mg per mL of composition.

According to the invention, myriocin or a composition according to any one of the embodiments and application described in the present description is administered to a subject requiring it at a concentration of myriocin per unit dose comprised between approximately 0.012 and 0.6 mg/die, preferably between 12 and 120 mg/day, in particular between 6 and 60 micrograms/day per eye. Under unit dose, in the context of the present invention, the dose is meant which is administered each time to the subject, be it divided into several administrations during the day, be it daily, or be it administered at intervals of days.

According to a preferred aspect of the invention, the compositions according to any one of the embodiments described in the present description comprise myriocin as unique active ingredient.

According to an alternative aspect of the invention, the compositions described in the present description can also comprise, apart from myriocin, even one or more different active ingredients for use in the surgical treatment of glaucoma, for example one or more topical antihypertensive or hypotonizing drugs.

Still, according to an additional aspect of the invention, the treatment of glaucoma, in particular the surgical treatment of glaucoma, can be performed by administration of myriocin or a composition according to any one of the herein described variants in association with other drugs, like the drugs conventionally used for the treatment of glaucoma, such as for example antihypertensive or hypotonizing drugs, at therapeutically effective doses to a patient requiring it.

Preferably, myriocin or the compositions according to any one of the embodiments described in the present description can be administered to a subject requiring it, optionally in combination to one or more additional active ingredients, such as for example antihypertensive or hypotonizing drugs, topically and/or intraocularly, for example by subconjunctival route. Preferably, myriocin or the compositions according to any one of the embodiments described in the present description are administered intraocularly. According to an additional aspect, myriocin or the compositions according to the present invention can be administered systemically, in particular intraperitoneally.

Myriocin or the above-mentioned compositions can be formulated in any form considered suitable by the person skilled in the art for applications of ophthalmic type, for example in any form suitable for direct topical application on the ocular surface.

According to an aspect of the invention, a composition according to any one of the variants described in the present description is in a form selected from the group consisting of: solution, suspension, gel, ointment, ocular drops, eye drops, spray, cream, pomade, foam, powder. Such compositions could be prepared according to any one of techniques known to the person skilled in the art.

In a preferred aspect according to the invention, the composition according to any one of the variants described in the present description is in form of eye drops. Such formulation provides a simple and not invasive administration mode, then encouraging the compliance of a patient who requires the treatment.

According to an aspect of the invention, the eye drops can comprise additional salts or excipients selected among sodium chloride, hydroxyethylcellulose, hyaluronic acid, and benzalkonium chloride.

In a preferred embodiment, the composition comprising myriocin according to any one of the herein described variants in form of eye drops comprises the following excipients:
0.9% sodium chloride
0.5% hydroxyethylcellulose
0.1% hyaluronic acid
0.01% benzalkonium chloride, wherein the percentages are expressed by weight with respect to the total volume of the composition, in particular g/100 mL of the composition.

An embodiment according to the invention in particular relates to a composition in form of eye drops for use in the treatment of glaucoma or to assist in the treatment of glaucoma, in particular the surgical treatment of glaucoma, according to any one of the variants described in the present description, comprising lipidic nanoparticles loaded with myriocin according to any one of the previously mentioned embodiments for carrying the compound at ocular level. This formulation allows to guarantee a good availability of myriocin at ocular level, as well as a controlled release and a high concentration of the compound.

In a preferred embodiment, the eye drops comprises myriocin between 0.01 and 0.5% by weight with respect to the total volume of the composition.

In a particularly preferred embodiment, the eye drops comprise myriocin, preferably in form of lipidic nanoparticles loaded with myriocin in an amount comprised between 0.01 and 0.1% by weight with respect to the total volume of the composition (i.e. gr/100 mL of composition). By pure way of example, the composition comprises in particular lipidic nanoparticles loaded with 0.68 mg/ml or 1 mg/ml of myriocin.

The present invention also relates to myriocin or a composition comprising it according to any one of the variants described in the present description, for use in the treatment or to assist in the treatment of a glaucomatous subject requiring it or who is undergoing surgical therapy of glaucoma.

In particular, myriocin or a composition comprising it according to any one of the embodiments described in the present description can be administered to said glaucomatous subject, pre-operatively, intra-operatively and/or post-operatively, that is before, during and/or after a surgical operation or therapy specific for the treatment of glaucoma.

Advantageously, the administration of myriocin or a composition according to the invention pre-, intra- and/or post-operatively is capable of explicating an action assisting in the treatment of glaucoma, thanks to the anti-inflammatory, antifibrotic and anti-cicatricial action exerted by the compound directly in the application site at ocular level.

Preferably, myriocin or a composition comprising it according to any one of the embodiments described in the present description, are administered to a subject suffering from glaucoma undergoing surgical therapy of glaucoma, after the surgical intervention, still more preferably both during, and after the surgical intervention, by any one of the forms or administration routes illustrated in the present description.

According to an aspect of the invention, the surgical therapy of glaucoma comprises any method of surgical treatment of glaucoma known to a person skilled in the art, in particular a parasurgical treatment, an incisional surgery treatment, in particular trabeculectomy, or a method of implantation of drainage devices.

According to an aspect of the invention, said myriocin or composition comprising it is administered to said subject undergoing surgical therapy of glaucoma, in particular a human subject, in a therapeutically effective amount.

The present invention also relates to myriocin or a composition comprising it according to any one of the embodiments described in the present description, for use in a method of treatment or a method to assist in the treatment of glaucoma, which method comprises at least a step of administering a therapeutically effective amount of myriocin or said composition to a subject requiring it, in particular a human subject. According to a preferred aspect of the invention, said method is a method of surgical treatment of glaucoma.

A method for the treatment of glaucoma is also herein described, in particular a method of surgical treatment of glaucoma, or a method to assist in the treatment of glaucoma, comprising the administration to a subject requiring it an effective amount of myriocin or a composition comprising it as described in the present description.

In any method of treatment or surgical therapy as described in the present description, the exact dosage and the administration frequency of myriocin or a composition comprising it will depend upon the particular seriousness of the condition to be treated, as well as upon age, weight and the general physical conditions of the particular subject, as it is well known to an expert skilled in the art.

As previously mentioned, a glaucomatous subject suitable to be treated with myriocin or with any composition comprising it according to the invention, is a subject suffering from any form of glaucoma, for example, congenital glaucoma, open angle glaucoma, closed angle glaucoma or glaucoma without hypertension.

According to an aspect of the invention, the administration of myriocin or a composition comprising it according to the invention can be performed in several unitary doses administrable at suitable time intervals, preferably in one or more daily doses, by using the same or a different administration mode.

By pure way of example, the following dosages could be administered: between 3 and 30 micrograms per eye, twice a day, or between 6 and 60 micrograms per eye, once a day, for a total of myriocin comprised between 12 and 120 micrograms per day.

### Examples

Some not limiting examples of the compositions according to the present invention are shown hereinafter. It is declared that the examples reported below were performed on commercially available cells.

### Example 1—Comparative Tests of Anti-Fibrotic Activity of Myriocin VS Mitomycin C

The anti-fibrotic activity of myriocin, compared to that of mitomycin C, was evaluated in vitro by using human dermic fibroblasts of healthy subjects, available commercially.

### Culture of the Fibroblasts

The fibroblasts, cultivated in culture medium DMEM (EuroClone) additioned with 10% of Fetal Bovine Serum (EuroClone) and 1% of penicillin/streptomycin (Sigma-Aldrich), were kept in incubator at 37° C., 5% of CO₂ until reaching the full confluence which corresponds approximately to 1.2-1.3*10⁶ cells. After detachment from the culture flasks (T75) with a solution of trypsin/EDTA, 1.5-2.5*10⁵ or 4.0*10⁵ fibroblasts were respectively plated in 60-mm or 100-mm wide Petri dishes for the subsequent treatments.

### Treatment of the Fibroblasts

The cells were divided into six experimental groups:
1. control group without treatment (denominated ctr)
2. group treated with TGFβ (denominated TGFβ)
3. group treated with Myriocin and TGFβ (denominated Myr/TGFβ)
4. group treated with Mitomycin C and TGFβ (denominated Mito/TGFβ)
5. group treated with Myriocin (denominated Myr)
6. group treated with Mitomycin C (denominated Mito)

### Experimental Protocol

On the day of treatment, in groups ctr, TGFβ, Myr, Myr/TGFβ the culture medium was replaced by fresh medium. The groups Myr and Myr/TGFβ were pretreated for 1 hour with myriocin 5 mM (Sigma-Aldrich) so as to obtain a final concentration of 50 μM. Ten minutes before the end of time of pretreatment with myriocin, in the groups Mito and Mito/TGFβ the medium was replaced by fresh medium containing mitomycin C 50 μM, corresponding to the maximum concentration suggested by the producer (Sigma-Aldrich), equal to 20 μg/ml. The pretreatment with mitomycin C, lasting 5 minutes to simulate the administration time in the operating room of the drug on the patient suffering from glaucoma, was interrupted by two washes, each one lasting 2 minutes, with phosphate buffer (PBS). To the groups TGFβ, Myr/TGFβ and Mito/TGFβ, TGFβ 200 ng/ml is then added to obtain a final concentration of 5 ng/ml, as reported in literature (Wu N. et al., Mol Cell Biochem. 2020; 470:175-188). The fibroblasts were then put in incubator for 24 hours, until performing the different analyses.

### Experimental Protocols for: Scratch Test, Trypan Blue Assay, Mass Spectrometry, Analysis in RT-PCR, Western Blotting.

### Scratch Test Assay

In six-well plates, 150,000 fibroblasts/well were plated and left to proliferate until reaching a confluence of 80-90%. At this point, defined time zero (T0), the cellular monolayer is damaged by creating a vertical groove corresponding to the well diameter. The fibroblasts, divided into treatment groups ctr, Myr and Mito, were observed at microscope 24 (T1) and 48 (T2) hours after the damage and after start of the treatments in order to evaluate the migratory capability and then the regenerative-fibrotic activity of the fibroblasts.

### Trypan Blue Assay

The fibroblasts, cultivated in ninety-six-well plates, were divided into the experimental groups: ctr, Mito and Myr. 24 hours after sowing (T0), the controls were collected for counting the live and dead cells and one proceeded with the treatment of the groups Mito and Myr for the subsequent 24 hours, with the respective substances. After detachment with a solution of trypsin/EDTA, the fibroblasts were isolated by centrifugation (100 g×6 minutes) and the obtained precipitate was resuspended in phosphate buffer. To the cellular suspension, 10% of 0.4% Trypan Blue dye solution was added. The live cells and the dead cells, coloured blue for the intracellular permeation of the dye through the damaged plasmatic membrane, are counted by using Bürker camera (Fabiani C., et al., Naunyn-Schmiedebergs Arch. Pharmacol. 2017; 390:741-751).

### Analysis in Mass Spectrometry of the Ceramide Content

After detachment with a solution of trypsin/EDTA, the fibroblasts were isolated by centrifugation (100 g×6 minutes). The obtained precipitate was resuspended in phosphate buffer (PBS), centrifuged at 100 g×6 minutes, resuspended again in phosphate buffer (PBS) and centrifuged at 500 g×5 minutes at 4° C. The precipitate was resuspended in 100 μl of PBS containing a mixture of protease inhibitors (Protease Inhibitor Cocktails, EDTA-free, Roche) and kept at −20° C. until use. The extraction of the lipids and the analysis of the samples for evaluating the ceramide content, whose synthesis is locked by inhibiting the enzyme serine-palmitoyl transferase by Myr, follow published protocols (Morano C. et al., Metabolites. 2022; 12 (5): 450).

### Analysis in RT-PCR

After detachment with a solution of trypsin/EDTA, the fibroblasts were isolated by centrifugation (100 g×6 minutes). The obtained precipitate was resuspended in phosphate buffer (PBS), centrifuged at 100 g×6 minutes, resuspended again in phosphate buffer (PBS) and at last centrifuged at 500 g×5 minutes at 4° C. The extraction of RNA (ReliaPrep™ Miniprep RNA extraction System, Promega), the retrotranscription in cDNA (GoScript Reverse Transcription Mix, Promega) and the analysis of the expression of the transcripts of interest by RT-PCR (SYBR Green system, Takara) take place according to published protocols (Zulueta A. et al., Cell Signal. 2018; 51:110-118).

The following transcripts of interest were analysed:
αSMA (α Smooth Muscle Actin): marker of the activation of the fibroblasts to miofibroblasts;
CTGF (Connective Tissue Growth Factor): protein of the extracellular matrix with pro-fibrotic activity;
MMP-9 (Matrix metalloproteinase 9): enzyme which degrades the proteins of the extracellular matrix.

### Analysis in Western Blotting

After detachment with a solution of trypsin/EDTA, the fibroblasts were isolated by centrifugation (100 g×6 minutes). The obtained precipitate was resuspended in phosphate buffer (PBS), centrifuged at 100 g×6 minutes, resuspended again in phosphate buffer (PBS) and centrifuged at 500 g×5 minutes at 4° C. The extraction of the proteins was performed by RIPA buffer, the dosage and the analysis in Western Blotting of the proteins of interest according to published protocols (Fabiani C., et al., Naunyn-Schmiedebergs Arch. Pharmacol. 2017; 390:741-751). 20 μg of total protein extract for each sample were loaded on 10% acrylamide/bis-acrylamide gel. The protein content of αSMA was analysed by using as primary antibody anti αSMA (Abcam, ab124964), diluted 1:1000 in TRIS buffer. The acquisition of the images and the quantification of the density of the obtained bands was performed by means of Alliance UVITEC instrument (Cambridge, UK).

### Results

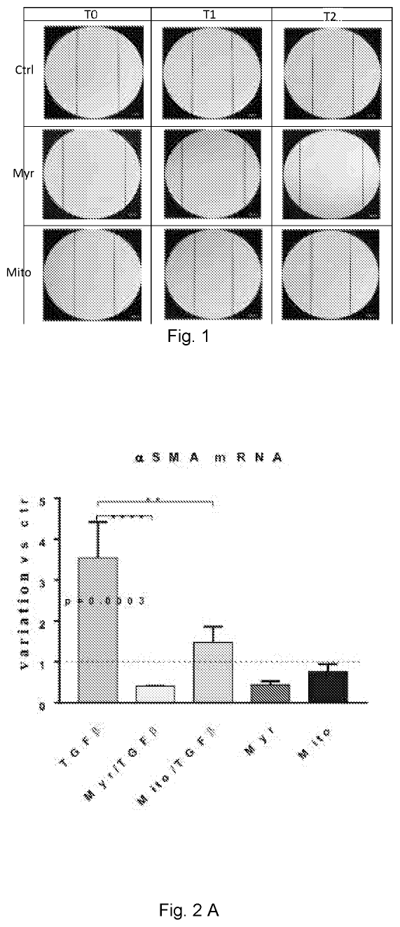

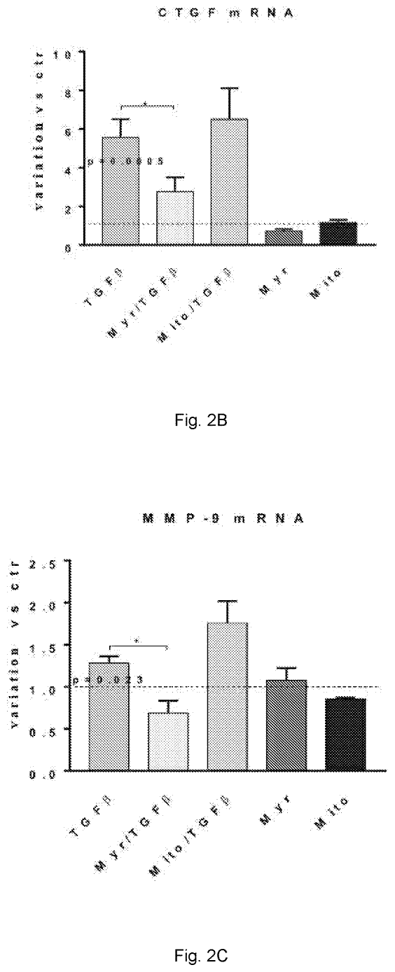

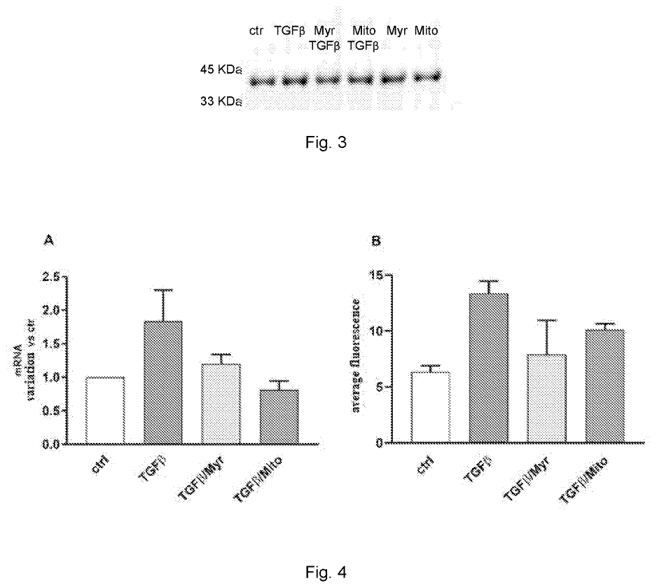

Myriocin showed anti-fibrotic efficacy on an in vitro model of the fibroblasts activated with TGFβ comparable to mitomycin C, but with a better cellular tolerability. After damage of the cellular carpet to stimulate the surrounding fibroblasts to migration and then to damage regeneration, the use concentration (50 μM) and treatment duration (24 and 48 hours) being equal, both substances reduced the migration of fibroblasts but mitomycin C, differently from myriocin, induced a progressive cellular death, as highlighted in FIG. 1.

The phenotypic data found confirmation in the results of the assay with Trypan Blue which allows to calculate the ratio between live and dead cells. After 24 hours, the control fibroblasts doubled (from 4200 to 8666 total cells/well), the ones treated with mitomycin C halved (from 10466 to 5466 total cells/well) whereas the ones treated with myriocin increased by about 70% (from 6800 to 11733 total cells/well). Myriocin is a specific inhibitor of the enzyme serine-palmitoyl transferase which controls the production of ceramide, key reaction of the synthesis of sphingolipids. As proof of the specificity of the performed treatment, the content of ceramide and of its immediate precursor dihydroceramide was evaluated by mass spectrometry, in the fibroblasts of the experimental groups ctr, TGFβ, Myr e Myr/TGFβ. The fibroblasts activated with TGFβ almost doubled the amount of ceramide and of the precursor with values ranging from 6.73 to 11.97 μmol/μl and from 0.74 to 1.16 μmol/μl, respectively. Myriocin reduced by about 15% and 25% the levels of ceramide and dihydroceramide with respect to the basal ctr whereas it lowered them, respectively, approximately by 50% and 60% with respect to the group stimulated with TGFβ. The analysis of the transcripts of some among the genes involved in the fibrogenetic process (αSMA, CTGF, MMP-9) highlighted that myriocin results to be more effective than mitomycin C in reducing the expression increase induced by the stimulation with TGFβ (FIG. 2). The protein expression of αSMA, evaluated by Western Blotting technique, follows the course of the transcript in the different experimental groups (FIG. 3).

Additional Data Supporting the Control of Transcription (Panel A) and the Translation (Panel B) of Collagen by Myriocin with Respect to Josamycin:

To further support what illustrated above, the inventors of the present patent application obtained experimental data showing that Myriocin is capable of controlling the transcription (panel A) and the translation (panel B) of collagen (FIG. 4).

The comparative analyses of the data obtained with Myriocin with respect to those obtained with Josamycin show that after administration of Josamycin 69.4 μg/ml (JM69.4), near to the effective concentration (JM75), the proliferation percentage of the fibroblasts is less than 50% whereas the data obtained by the inventors (also reported in table 1 hereinafter), obtained with proliferation/vitality MTT assay, show that after 24 hours of treatment with Myriocin 50 μM, the relative proliferation/vitality index is higher than 60%. These proliferation data result to be an improvement with respect to those obtained by the authors with JM47.2, which is equivalent to a concentration of approximately 56 μM, not considered among the effective concentrations in anti-fibrotic sense.

TABLE 1

| | | | | | | | | % | % |
|---|---|---|---|---|---|---|---|---|---|
| | | | | | | | | proliferation | proliferation |
| NT | | | | MYR 24 H | | | | NT | MYR 24 H |
| 133.24 | 162.49 | 181.84 | 206.09 | 126.35 | 105.21 | 103.88 | 88.09 | 100% | 62% |

NT: not treated fibroblasts; MYR 24 H: fibroblasts treated 24 hours with Myriocin 50 μM.
Table shows the values of absorbance at 540 nm (4 replications for the two experimental groups: NT and MYR 24 H). The average of the four values for the two experimental group and the proliferation/vitality percentage was calculated, considering as 100% the proliferation value of the not treated fibroblasts after 24 hours.

The comparison of the obtained results demonstrates that the use of Myriocin shows an unexpected improvement with respect to Josamycin, absolutely not predictable based upon what described in the prior art.

1-11. (canceled)

12. A method of treating glaucoma or assisting in the treatment of glaucoma, the method comprising administering myriocin in a therapeutically effective amount to a glaucomatous subject.

13. The method of claim 12, wherein the subject is a glaucomatous subject undergoing surgical therapy of glaucoma.

14. The method of claim 13, wherein the myriocin is administered to the subject pre-operatively, intra-operatively, and/or post-operatively.

15. The method of claim 12, wherein the myriocin is administered in a unit dose between 0.012 and 0.6 mg/day per eye.

16. The method of claim 12, wherein the myriocin is administered in a unit dose between 6 and 60 micrograms/day per eye.

17. The method of claim 12, wherein the myriocin is administered topically and/or intraocularly.

18. The method of claim 12, wherein the myriocin is administered in a composition comprising the myriocin and one or more carriers, and/or diluents, and/or excipients.

19. The method of claim 18, wherein the myriocin is present in the composition in a concentration between 0.1 and 5 mg per mL of the composition.

20. The method of claim 18, wherein the myriocin is present in the composition in a concentration between 0.1 and 1 mg/mL of the composition.

21. The method of claim 18, wherein the composition is in a form selected from the group consisting of a solution, a suspension, a gel, an ointment, ocular drops, eye drops, a spray, a cream, a pomade, a foam, and a powder.

22. The method of claim 18, wherein the composition is in a form of eye drops.

23. The method of claim 18, further comprising one or more additional active ingredients for treating glaucoma.

24. The method of claim 23, wherein the one or more additional active ingredients comprise an antihypertensive drug, a hypotonizing drug, or a combination thereof.

25. The method of claim 18, wherein the composition comprises one or more carriers and the one or more carriers comprises solid lipid nanoparticles.

26. The method of claim 12, wherein:
the myriocin is administered in a unit dose between 6 and 60 micrograms/day per eye;
the myriocin is administered topically and/or intraocularly;
the myriocin is administered in a composition comprising the myriocin and one or more carriers, wherein the one or more carriers comprises solid lipid nanoparticles; and
the myriocin is present in the composition in a concentration between 0.1 and 1 mg/ml of the composition.

27. The method of claim 26, wherein the subject is a glaucomatous subject undergoing surgical therapy of glaucoma.

28. The method of claim 26, further comprising one or more additional active ingredients for treating glaucoma, wherein the one or more additional active ingredients comprise an antihypertensive drug, a hypotonizing drug, or a combination thereof.

29. The method of claim 28, wherein the subject is a glaucomatous subject undergoing surgical therapy of glaucoma.
